(19) United States
(12) Patent Application Publication
(10) Pub. No.: US 2009/0270945 A1
(43) Pub. Date: Oct. 29, 2009

(54) Portable Applicator for Collagen Stimulation

(76) Inventors: Ernestine Binder Markoll, Andratx (ES); Richard Markoll, Andratx (ES)

Correspondence Address: THE NATH LAW GROUP, 112 South West Street, Alexandria, VA, 22314, US

(21) Appl. No.: 12/225,165

(22) PCT Filed: Mar. 16, 2006
(86) PCT No.: PCT/EP2006/002421

§ 371 Date: Sep. 16, 2008

(51) Int. Cl. A61N 1/00

(52) U.S. Cl. 607/50

(57) ABSTRACT

A portable applicator for pulsating signal therapy comprises a support body (10), and at least two signal-generating arrangements (30) that are arranged in or on the support body (10). The at least two signal-generating arrangements (30) are connected in series, and each of the at least two signal-generating arrangements (30) comprises at least two signal-generating units (20), which are connected in parallel.

### FIELD OF THE INVENTION

The invention concerns a portable applicator to be used for pulsed signal therapy in the treatment of skin changes, due to, for example, skin ageing or burn-related injuries, and especially a portable applicator for the use in pulsed signal therapy for the stimulation of collagen regeneration.

It is therefore essential that the pulsed magnetic fields be applied on the correct body points in appropriate strength and in the proper frequency for a certain period of time, so that the collagen-containing tissue of the body is stimulated.

### BACKGROUND OF THE INVENTION

The human skin consists of several layers: the cuticle (epidermis), the corium (dermis) and the hypodermis (subcutaneous tissue). The skin is bordered by the general body fascia, which consists of collagen fibres. After the fascia follows—depending on the body region—either the musculature, the bones, cartilage or fat. The corium is an elastic skin layer, which is composed mostly of the structural proteins collagen and elastin. It contains, among other things, a dense network of collagen fibres, which are filled with elastic connective tissue. Cartilage is a supporting tissue, which consists of cartilage cells (chondrocytes) rich in water and intercellular substances. The extra-cellular matrix of the cartilage consists of 60 to 80% water. The intercellular substance is composed of basic substances (proteoglycanes and glucoproteins) and fibres (collagens). Proteoglycans, glucoproteins and collagens are produced by chondrocytes. The collagens build a network, wherein proteoglycans and glucoproteins are stored and fixated.

Several types of collagens exist. All collagens have in common the fact that they are built out of three polypeptide chains, which are twined around each other in the form of a triple helix. Collagen molecules of the type I, II, III, V and VI, which are called “fibrillar collagens”, have similar molecular structures. Collagen molecules of the type IX, XII and XIV, so-called “fibril-associated collagens” contain multiple triple helix domains, which are intermitted by non-collagen domains. The several types of collagen molecules are significantly different. Collagen molecules of type V have a low concentration of alanine, but a high concentration of alkaline amino acids. They also align themselves with tetramers. Type VIII is a strong protease. Type IX is found on the surface of collagen type II. Collagen molecules of type XI are short triple helices with very long globular elongations. Collagen molecules of type VII have an N-terminal area of domain with three fingers, similar to the collagen types XII and XIV.

The structure of the different skin layers changes with increasing age. The respective skin layers get thinner, the fat layers abate, and the structural proteins of the corium collapse; these changes are visible as wrinkles.

In the current state of technology, there are known processes for the treatment of wrinkled or aging skin, such as the discoloration of skin through magnetic field therapy. This process is described, for example, in U.S. Pat. No. 5,669,868, which names R. Markoll as the inventor.

During this process, the skin is exposed to a magnetic field of less than 20 gauss. The magnetic field is produced by a pulsed signal with a rectangular waveform. The production of collagen is stimulated by the application of the magnetic field. In U.S. Pat. No. 5,669,868 the magnetic field is produced through an annular coil, inside of which the body part to be treated is positioned.

### SUMMARY OF THE INVENTION

One of the challenges of the invention is to provide a portable applicator for usage in pulsed signal therapy that allows for a more effective stimulation of collagen regeneration than the devices already available.

This challenge is solved by the portable applicator with the features described in claim 1.

The invention is based on the idea that an increased area of treatment be provided precisely on the points where stimulation of collagen regeneration is necessary.

According to this aspect, the present invention comprises a portable applicator having a support body and at least two signal-generating arrangements, which are placed in or on the support body. These at least two signal-generating arrangements are connected in series, and each of the two arrangements comprises at least two signal-generating units, which are connected in parallel.

Through the applicator, and in accordance to the invention, an increased field of treatment can be provided precisely to the parts of the body where collagen regeneration is necessary. The body parts in need of treatment do not have to be placed directly between the coil and an “opposing pad” is not necessary. This means that two opposing coils—in between which the body part to be treated would be placed—are no longer needed. The applicator of the present invention enables therefore a more effective and targeted usage of the magnetic fields on the relevant body parts.

Furthermore, advantageously the portable applicator can be easily transported, in a case or trolley, for example. For the appropriate fixation on the desired body part, the applicator may feature additional elements for its fixation, such as straps, belts, adhesive tapes, hook-and-loop fasteners and so on.

Each signal-generating arrangement of the portable applicator comprises preferably three signal-generating units connected in parallel. A parallel connection of three signal-generating units is especially beneficial, because this number permits an optimal field distribution between the signal-generating units, which assures an optimal use of the applicator.

Cored coils are preferably used as signal-generating units. Optimally, the coils should be aligned perpendicularly with their longitudinal axis to the surface structure of the supporting body, to assure a large depth of penetration of the magnetic field into the skin.

In accordance with a preferred embodiment, the signal-generating units produce a pulsating magnetic field of fewer than 25 gauss (G), preferably of 10-20 gauss. With field strengths of this order it can be assured that the magnetic field is strong enough to penetrate the fat layer down to the cartilage. Thus the cartilage cells and the (connective) tissue are effectively stimulated by the applied magnetic field and the production of collagen is automatically co-stimulated.

The signal-generating units will preferably be imbedded in a protective body. A preferred embodiment sees the protecting body being placed directly over the signal-generating units in the form of a cover. In another preferred embodiment, the protecting body is built as a cap, which covers the signal-generating units.

Preferably, the protective body will be enfolded in a coating preferably made of a biologically compatible material. A coating—that is, the part of the portable applicator that touches the body directly—made of a skin-friendly material is particularly preferable. Most preferably, the coating should be made out of Neoprene®. According to another preferred embodiment, the coating is made by latex.

The support body will preferably be made of an insulating gel in which the signal-generating arrangements are imbedded. The use of a gel provides an optimal adaptability to the form of the body parts to be treated.

According to a preferred embodiment, the support body will be malleable. Preferably, the support body will be adaptable to and assume the form of the body part to be treated. It is considered to design the support body in a way as to be adaptable to the treatment of the neck and upper chest areas.

According to another preferred embodiment, the support body is shaped as a facemask. By designing the applicator as a facemask, the wrinkled or wrinkle-prone areas of a person can be directly exposed to the magnetic field, thus achieving a particularly efficient stimulation of collagen regeneration.

Preferably, the support body is built in several parts. A preferred embodiment sees the support body as two symmetric parts. In an especially preferred embodiment, the several parts of the support body are flexible and/or elastically connected with each other. Thereby an optimal fitting of the mask would be assured, regardless of different facial shapes of various persons. Furthermore, the flexible or elastic connection of the parts of the support body would improve its handling. The mask, for example, could be bended or folded, so that it does not take up too much space in transportation or storage.

One of the uses of the invention is characterized by the features set forth in claim 12. This aspect of the invention concerns especially the usage of the applicator described above for the cosmetic treatment of skin. Cosmetic treatment comprises wellness and anti-ageing treatment as well as the cosmetic treatment of wounds and scars.

Another possible use of the invention is set forth by the features of claim 13. This aspect of the invention concerns the use of the applicator described above in the therapeutic treatment of skin. Therapeutic treatment of skin encompasses, for example, the treatment of wounds and scars for therapeutic purposes, by accelerating or advancing the healing of wounds or the formation of smaller or “smoother” scars.

Preferably, the regeneration of collagen of types II, IX, XI and X is beneficially stimulated by the use of the applicator. The regeneration of these collagen types contributes particularly to the improvement of the external appearance of the skin.

In accordance with the invention, a device is characterised in claim 15. According to this further aspect of the invention, a device for pulsed signal therapy is provided, which comprises the applicator described above and a device for the feed of the control signal (control module) to the signal-generating units. The applicator and the control module should preferably be provided as a single, compact unit, i.e. the applicator is firmly connected with the control module and/or the applicator and the control module are provided as an integral unit within a common housing. This unit can be placed in a case or on a trolley for ease of transportation. Another possibility is for the control module to be detachable from the applicator. In addition, in this possible design, the device may be provided in a case or trolley for ease of transportation.

Preferably, the control signal will be a constant flux voltage with a maximal rate of 30 pulses per second, especially 1 to 12 pulses per second more preferably 5 to 12 per second, and a rectangular pulse form with an abrupt increase and abrupt plunge in voltage. The voltage is preferably 24 V.

Beneficially, the control module will reduce the pulse width throughout the period of operation and increase the pulse amount throughout the period of operation.

Further beneficial embodiments and improvements of and to the invention are described in the dependent claims. The invention includes also designs that are combinations of features, listed separately in the claims, figures and description of the invention.

### SHORT DESCRIPTION OF THE FIGURES

In sequence, the invention is described in a purely exemplary fashion, with attached figures, in which:

FIG. 1 is a schematic depiction of a first embodiment of the applicator, according to the invention;

FIG. 2 is a schematic depiction of the wiring of the first embodiment described in FIG. 1;

FIG. 3 is a schematic depiction of a second embodiment of the applicator, according to the invention;

FIG. 4 is a schematic depiction of a modification to the second embodiment;

FIG. 5 is a schematic depiction of the wiring of the second embodiment of the applicator, seen in FIG. 4, according to the invention;

FIG. 6 is a lateral view of the third embodiment of the applicator, according to the invention;

FIG. 7 is a top view of the third embodiment of the applicator, according to the invention;

FIG. 8 is a schematic depiction of the wiring of the third embodiment, described in FIG. 7

### DETAILED DESCRIPTION OF THE PREFERRED DESIGNS

According to the invention, the portable applicator comprises a support body 10 and at least two signal generation arrangements 30, which are placed in or on the support body 10, as is shown, particularly in the example of FIG. 1. The at least two arrangements of signal generation 30 are connected in series, and each of the at least two arrangement of signal generation 30 comprises at least two signal-generating units 20, which are connected in parallel. Through the use of the two arrangements for signal generation 30 connected in series, which, again, include at least two signal-generating units 20 each, connected in parallel, a more effective stimulation of collagen regeneration is achieved than with the methods currently in use. The applicator, in accordance with the invention, allows for am increased treatment field—that is, a more tightly focused or localised and more intensive magnetic field—can be applied on the precise points on which the stimulation of collagen regeneration is required.

Preferably, the magnetic fields of the single signal-generating units 20 overlap each other, whereby the distance between the single signal-generating units is adapted to their respective usage. Because of the overlap, the individual magnetic fields will be amplified, and it becomes possible to produce a strong magnetic field within a small space with signal-generating units of small dimensions. The invention is however not restricted to a use whereupon the magnetic fields of the signal-generating units 20 must overlap each other. When and if appropriate, the respective signal-generating units 20 can be placed far away from each other in such a way as that the magnetic fields do not overlap. In general, the signal-generating units 20 are placed further away from each other during an extensive treatment of the skin and closer together during a punctual treatment. An extensive treatment of the skin can be considered for the cosmetic or therapeutic treatment of scars, for example. A scar can be seen as a single “cell” and its treatment will require a broader magnetic field, that is, a less concentrated magnetic field. Embodiment 1 of the present invention, described below in more detail, is an applicator particularly suitable for extensive treatments.

On the other hand, when there are many “cells” within a small area, such as below the eyes, a punctiform magnetic field, that is, a very concentrated magnetic field, is a more appropriate treatment. Embodiments 2 and 3, described below in more detail, are examples of designs for a concentrated or very targeted and intensive treatment, in accordance with the invention. These exemplary designs show that that the signal-generating units 20 can be placed according to the needs of the body part to be treated (see, for example, FIGS. 4, 6 and 7). For example, the signal-generation units 20 are placed very close together in the area below the eyes, because that area has many cells within a small space, whereas in the forehead, the signal-generating units 20 are positioned further away from each other, because this area contains a smaller concentration of cells.

The distance between the signal-generating units can therefore be adapted to the structural construction of the skin surface to be treated and therefore, to the particular treatment requirements. Furthermore, the distance of the signal-generating units can be chosen according to whether an overlap of the magnetic fields of the respective signal-generating units is desired, and how large the size of the overlap should be. When an overlap of the magnetic fields of the individual signal-generating units is desired, properties of the used signal-generating units such as the size and strength of the magnetic fields must be taken into account. The preferred distance between the individual signal-generating units will be between 0.5 times (e.g. for the area below the eyes) and 3 times the diameter of a signal-generating unit (e.g. the area of the forehead). A person skilled in the art expert will understand that the distance between single signal-generating units may vary, according to the requirements and type of coil used.

Below follows a series of detailed descriptions of preferred embodiments, in accordance to the invention and in relation to the attached figures.

### EMBODIMENT 1

FIG. 1 is a schematic depiction of a first design of the portable applicator, in accordance with the invention. In this design, the applicator is malleable (surface applicator). The surface applicator contains a support body 10 and a multitude of signal-generating units 20. A device to control the feed of the control signal of the signal-generating units is also provided, but not shown in FIG. 1. For appropriate fixation on the desired body parts, the applicator can be provided with the usual fasteners, such as straps, belts, adhesive tapes, hook-and-loop fastener and so on.

The support body 10 shown in FIG. 1 is built out of a flexible circuit board with a rectangular form. The material of the support body 10 and its form are not limited to the design shown in FIG. 1. The support body 10 can be built out of any suitable material and can be shaped into any suitable form, such as, for example, independence on the body part to be treated. The malleable support body 10 can be provided, for example, as a rectangle with rounded corners, an oval form, or a kidney-shaped form. The shape can be adapted to the body part on which the support body 10 is laid on or encloses, to assure an optimal application of the support body 10 on the respective body part. When the support body is made out of a hard material it can be built in several parts, to allow a better adaptability to the shape of the respective body part. These parts can then be connected to each other by means of joints or other arrangements, such as straps, hook-and-loop fasteners, etc. The malleable applicator can also be used on areas such as the neck and the upper chest.

The material out of which the support body (10) is constructed can be hard as well as flexible and/or elastic. Besides the flexible circuit board depicted in FIG. 1, also other rigid and/or stiff circuit boards, flexible and/or elastic materials and webs, malleable unities made of rubber as well as combinations thereof can be considered. Preferably, the support body will be made of Neoprene®. Other preferred materials for the support body 10 include latex and synthetic materials such as GRP, PP and/or PVC. Yet another possibility is to build the support body out of a gelatinous mass. This means the signal-generating units or the treatment coils will preferably be imbedded in a mask, which will preferably be shaped as the body part to be treated, which will be filled with an insulating gel electrically similar to the one used for the treatment of the eye area. In this case the support body will be especially adaptable to the shape of the body part to be treated. Depending on the material used for the support body 10, the signal-generating units 20 will be placed on the support body 10 or imbedded into the support body 10.

In order to better protect the signal-generating units 20 against external factors (mechanic, thermal and/or chemical), these units can be inside a protective body (not depicted) or, alternatively, surrounded by said protective body. The protective body could cover one or more signal-generating units 20 and be built, for example, in the form of a cap that is attachable to the signal-generating units 20. The term “protective body” comprises all materials suitable for the protection of the signal-generating units 20, including, for example, protective paint and other coating materials.

The support body 10 can be covered or, alternatively, coated. The coating (not depicted) of the support body 10 can be single-layered or multi-layered. The coating can consist, for example, of three layers, whereby an additional layer of a malleable material, adaptable to the body parts to be treated (for example fluid, gel, foam, etc) can be placed between an internal and an external covering layer. Suitable materials for the coating of the support body 10 include, but are not limited to: fabric, tissues, Neoprene®, rubber, synthetic materials such as GRP, PP and or/ PVC, as well as any combination of these materials. Since the coating will be in direct contact with the skin of the patient, the material of the coating should be particularly skin-friendly.

In principle there are two types of coil: air coils and core coils. Air coils have no magnetic conductive core to amplify the magnetic field and provide very little inductance. To conduct applicable inductances with low internal resistance, coils with a large wire cross-section are necessary, but these are not suitable to the purposes of the present application. Relatively small coils, with a core made of a magnetisable material (such as ferrite or iron powder) that achieves a large inductance are preferred for the use in wellness/anti-ageing applications as signal-generating units 20; this type of core multiplies the inductance of the coil.

The structural shape of the signal-generating unit 20 is preferably stationary/radial. Each individual coil may have, for example, the following characterising values: 10.000 μH, +/−10% tolerance at a test frequency of 10 kHz, max. 0.08A DC at 20° C. To ensure a large penetration depth of the magnetic fields down to the deeper skin layer, that is, down to the fascia, the coils should be oriented along their longitudinal axis vertically to the surface structure of the support body.

The device for the feed of the control signal (not depicted) to the signal-generating units 20 will preferably be a control module with the following technical data: entry 100-200 VAC, 50-60 Hz, power consumption max. 2.2A; exit 24 VDC, max. −30 Hz. The external design of the control module, that is, its frame, can be modelled to be adapted to specific needs. When the control module is portable, it may also feature an additional carrying handle, for example. In the case of a control module, which is planned to be integrated into a module unit together with other modules, its frame may comprise additional devices for its fixation in the module unit.

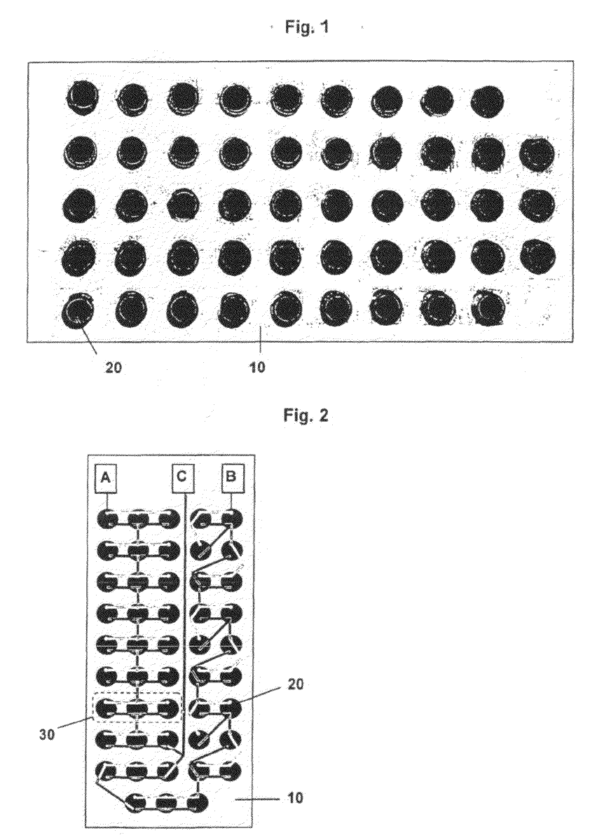

FIG. 2 is a schematic depiction of the wiring of the surface applicator shown in FIG. 1. The device for the feed of the control signal is also not depicted in FIG. 2. In this embodiment, forty-eight signal-generating units 20 are positioned on the support board 10. Three signal-generating units 20 form a signal-generating arrangement 30, so that—in this embodiment—there are a total of sixteen signal-generating arrangements 30 provided. The three signal-generating units 20 of a signal-generating arrangement 30 are connected in parallel, and all the signal-generating arrangements 30 are connected in series. Although this particularly preferred embodiment relates to the signal-generating arrangement 30 of three signal-generating units 20, a person skilled in the art will understand that the signal-generating arrangements 30 can have a different amount of signal-generating units 20. It is possible to conceive versions where the signal-generating arrangements 30 comprise two, four or more signal-generating units.

In the schematic depiction of FIG. 2, the surface applicator is divided in two connectable parts. On each side, a connection A or B is placed. A common connection C is also provided. Although it is shown in FIG. 2, the different parts do not need to have the same number of signal-generating arrangements 30. A connection cable for the electrical connection of the malleable applicator with the control module can—through the preferably existing coating of the support body—be added to any spot that is used for connections A, B and C on the support body 10.

In accordance with the invention, the signal-generating units 20 produce a magnetic field of fewer than 25 gauss in strength, preferably between 10-20 gauss. The strength of the resulting magnetic field is measured on the sides of the applicator, which are supposed to face the body, and decreases in relation to the distance to the applicator.

### EMBODIMENT 2

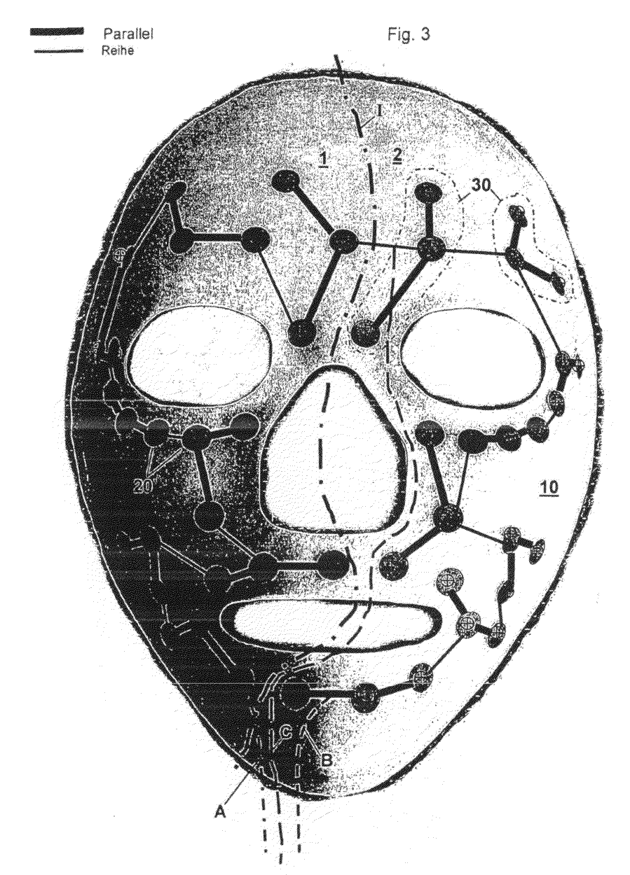

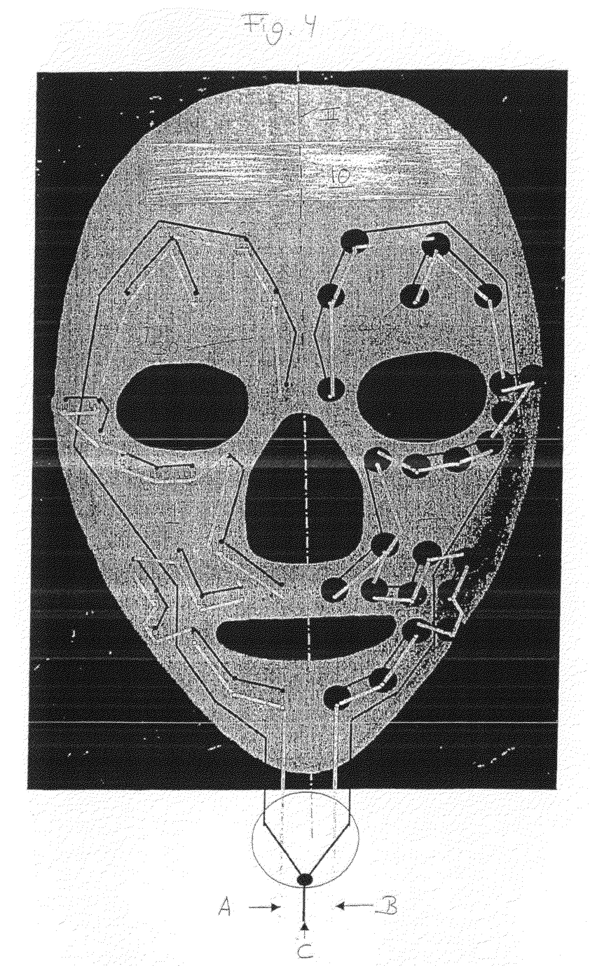

The design of the applicator shown in FIG. 3 is especially conceived for facial treatment, in accordance to the invention. Same and similar parts to those of embodiment 1, shown in FIGS. 1 and 2, are labelled with the same reference sign. Furthermore, only the differences between the two embodiments are described. Regarding the similarities between the two designs in terms of function and form, refer to the description of embodiments 1, above.

In the schematic depiction of embodiment 2 in FIG. 3 the applicator (facemask) features a support body 10 divided through a parting line I in a right face half 1 and a left face half 2. Preferably, each half 1, 2 of the facemask will have eight signal-generating arrangements 30 connected in series, each composed of three signal-generating units 20 connected in parallel. That means that each side 1, 2 of the facemask has 24 signal-generating units 20 and thus there are 48 signal-generating units 20 on the facemask in total. The signal-generating units are placed for the pointed stimulation of wrinkled or wrinkle-prone areas of the face. The arrangement of the signal-generating units 20 on the support body 10 is such that when the mask is applied, the signal-generating units 20 are positioned directly on the wrinkled or wrinkle-prone areas of the face of the person to treat.

The face halves 1, 2 depicted are unequal in size according to this embodiment, but they can also have the same dimensions (see, for example, FIG. 4). The facemask depicted in FIG. 3 is built as one piece. Possible materials for the support body and for an eventual protective body and/or coating have already been discussed in the description of embodiment 1.

The facemask depicted in FIG. 3, as well as other possible facemask designs yet to be discussed, will preferably be made of a synthetic material such as dyed, glass-reinforced plastic (GRP). The production may follow the monocoque technique, whereby the hollow space between the shells with the glued and connected signal-generating units 20, is filled with a single-component polyurethane (PU) foam, for the stabilisation of the form/shells. The preferred production processes of the masks include deep drawing and injection moulding.

The facemask depicted in FIG. 4 is a modification of embodiment 2, shown in FIG. 3. As is visible in FIG. 4, a parting line II is dividing the facemask right face half 1 and left face half 2. Preferably, both halves 1, 2 will have the same size, that is, the same surface area. However, the two face halves 1, 2 can also have different sizes. Several signal-generation units 20 will be positioned on each face half 1, 2. In the design depicted in FIG. 4, the signal-generating units 20 are arranged symmetrically on the support body 10 in relation to the parting line II. That means that each face half 1, 2 has eight signal-generating arrangements 30 comprising three signal-generating units 20 each, positioned symmetrically in relation to the parting line II. The arrangement of the individual signal-generating units 20 can be adapted to individual needs; an asymmetric arrangement or an arrangement with a different number of signal-generating units 20 on each face half 1, 2 is possible.

The mask shown in FIG. 4 is built in several parts, unlike the design depicted in FIG. 3. The mask depicted has two parts, but the number of parts of this design is not necessarily limited to two. The two parts of the mask—or both face halves 1, 2—can be articulated with each other, to assure an optimal fitting and an easy handling of the mask. The term “articulated”, as used here, includes all types of connections allowing a rotating, flapping, folding or pivoting of the different parts relative to each other, in one or several directions. Any known means of connecting possibilities can be taken in consideration for this end, including joints, hinges, pivot pins, etc. For the arrangement of multiple parts, not only articulated connections between single parts but—alternatively or in addition—also elastic connections such as bands, straps and other elastic agents or materials can be taken into consideration for the connection of the face halves in and/or on their interface area.

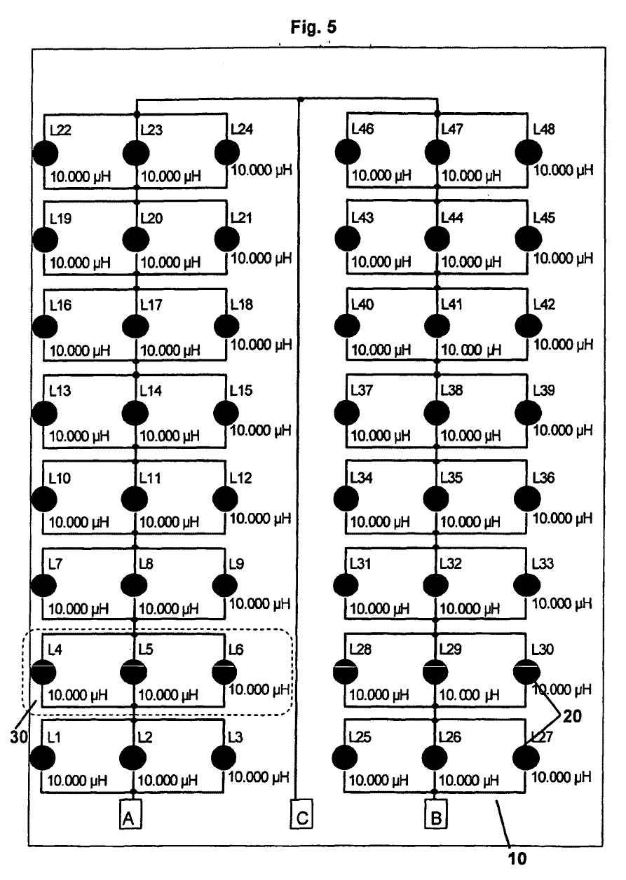

The facemasks of embodiment 2 depicted in FIGS. 3 and 4 comprise, in accordance with the invention, three connections A, B and C each, to provide a connection to a control module (not depicted). Connection A is provided for the signal-generating units 20 of the right face half 1 of the facemask. Connection B is provided for the signal-generating units 20 of the left face half 2 and Connection C is a common connection. FIG. 5 depicts a schematic example of the wiring of the facemask design depicted in FIG. 4, in accordance with the invention.

As is visible in FIG. 5, the facemask, in accordance with the invention, includes on each side 1, 2 of the mask eight signal-generating arrangements 30 connected in series, each with three signal-generating units 20 connected in parallel. At a parallel connection of three signal-generating units 20 the field distribution between the signal-generating units 20 is optimal. Furthermore, all three signal-generating units 20 of a signal-generating arrangement 30 have preferably the same inductance, so that each of the three signal-generating units 20 connected in parallel has the same voltage. As is shown in FIG. 5, all signal-generating units 20 positioned on the support body 10 have the same inductance. Therefore, the same amount of electricity flows on both face halves 1, 2 because there is an equal number of signal-generating units 20 on each face half 1, 2.

### EMBODIMENT 3

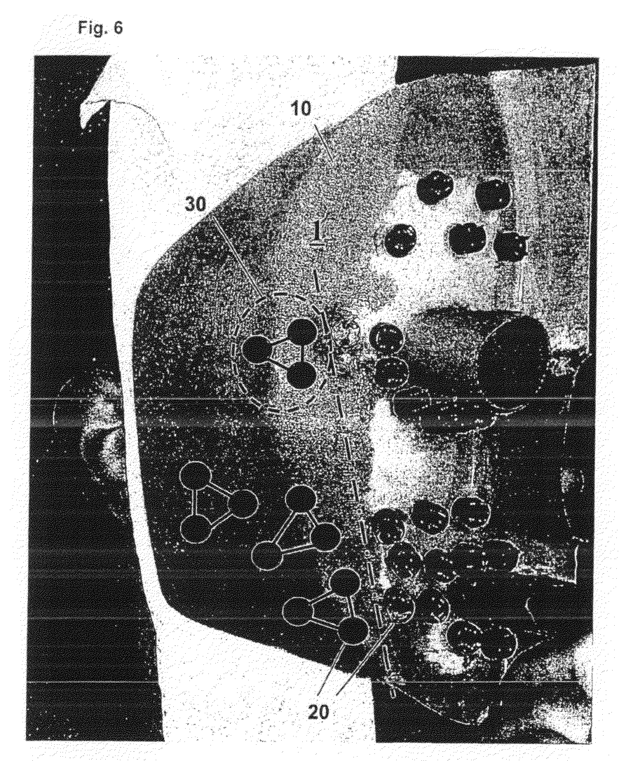

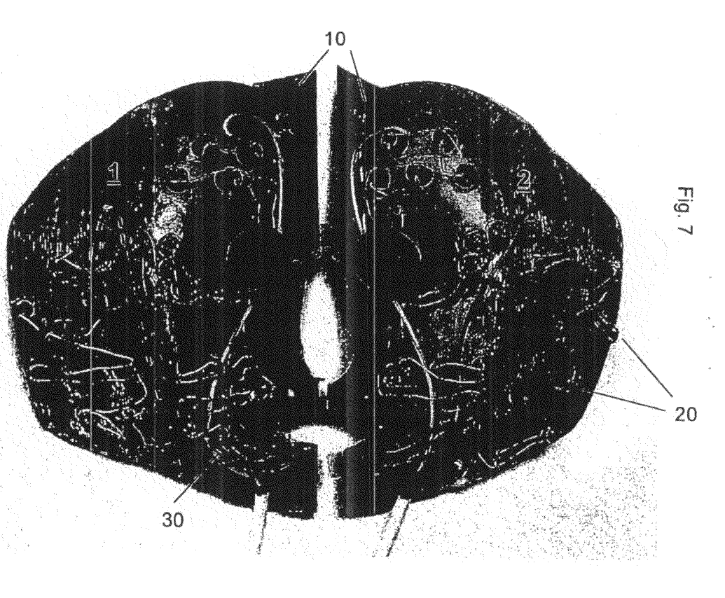

FIGS. 6 and 7 depict different views of embodiment 3 of the applicator, in accordance with the invention. Similarly to embodiment 2, embodiment 3 is also for facial treatment. However, the facemask of embodiment 3 is planned for a wider field of treatment, as will be described in more detail below. Same and similar parts to those of embodiment 2, shown in FIGS. 3, 4 and 5 are named with the same reference sign. Furthermore, only the differences between the two designs are described. Regarding the similarities between the two designs in terms of function and form, refer to the description of embodiment 2, above.

FIG. 6 depicts a lateral view of the facemask according to embodiment 3. In particular, FIG. 6 shows only the right half 1 of a mask, put on the face of a person. FIG. 7, on the other hand, depicts the facemask, in accordance with embodiment 3, in top view, and both face halves 1, 2 are shown. Similarly to embodiment 2, described above, embodiment 3 can also be built as one or several parts. Should a multipart mask (see FIG. 7) be chosen, the individual parts could be connected with each other by means of articulations and/or elastic elements (not depicted). By means of the multipart arrangement of the mask and of the articulated and/or elastic connections of the individual parts of the mask, its optimal fitting and easy handling can be assured.

Furthermore, a construction of the support body 10 of the right and left halves 1, 2 as several parts and using different materials can be taken into consideration. The external section of each face half 1, 2, on which 12 additional signal-generating units 20 are placed each, for example, could be built out of a different material than that used for the internal section of each face half 1, 2, on which the other 48 signal-generating units 20 are placed. Another possibility would be to build the internal or external section out of an elastic material, to fixate it then to the other section. This would assure an optimal fitting of the facemask without having additional articulated or elastic connections between the individual parts.

In addition to the 48 signal-generating units 20 of embodiment 2, embodiment 3 contains 24 additional signal-generation units 20. The additional signal-generating units 20 are—seen from the middle of the mask—outside the dotted lines in FIGS. 6 and 7. Four signal-generating arrangements 30 are positioned on each face half 1, 2 on a support body 10. The signal-generating arrangements 30 comprise three signal-generating units 20 each. Embodiment 3 of the applicator, in accordance with the invention, broadens the treatment field, so that the borders of the facial area may be exposed directly to a magnetic field, to achieve a more effective treatment.

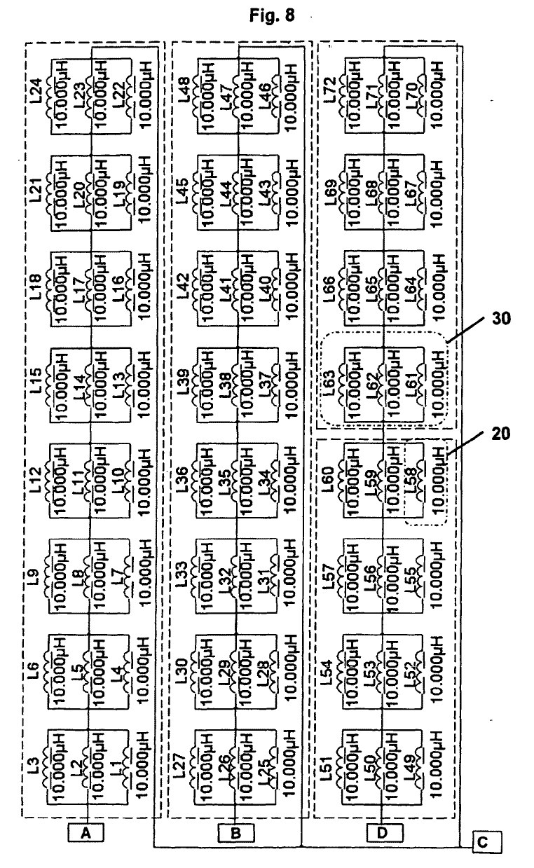

FIG. 8 depicts a schematic demonstration of the wirings of Design 3 shown in FIG. 7. As can be seen in FIG. 8, the facemask with a wider treatment field is divided into three connected sectors. As is the case with Design 2, Design 3 features also connections A and B for each part of the signal-generating arrangements 30. Each of these signal-generating arrangements 30 equates the amount of signal-generating arrangements 30 of the facemask of embodiment 2, that is, connections A and B are provided for the 48 signal-generating units 20. For the broader treatment field, a common connection C is provided for the other 24 signal-generating units 20, that is, 12 signal-generating units 20 on each face half 1, 2. The connection of these connection to the control module is not depicted.

### Usage of the Applicator According to the Invention

The description below of the usage of the portable applicator (or device) and its designs for the cosmetic treatment of skin is given merely in an exemplary fashion, in accordance with the invention.

The term “cosmetic treatment of skin”, as herein used, refers to wellness and anti-ageing treatments, as well as the cosmetic treatment of wounds and scars. The term “wellness treatment”, as herein used, refers to, among other uses, a cosmetic treatment of the skin to improve sensitivity of the skin and achieve a more lustrous complexion. The term “anti-ageing treatment”, as herein used, refers to, among other uses, a cosmetic treatment of the sin for the reduction of wrinkle depth and increase of the tightness and firmness of the skin. In general, a cosmetic anti-ageing treatment will produce a cleaner and tighter appearance of the skin. Furthermore, the cosmetic treatments of wounds and scars, as well as skin discoloration, are considered as uses of the applicator, in accordance with the invention. The term “skin discoloration” refers to the naturally occurring discoloration of skin, such as caused by, for example, liver spots, as well as skin discoloration resulting from trauma, such as haematomas, bruises or as a result of surgical or other operations. The cosmetic treatment is suitable for all ages. Older people, in particular, usually exhibit less muscle so that a maximal energy force can be transmitted to the body parts treated.

For the cosmetic treatment of skin, that is, for the stimulation of collagen regeneration, electromagnetic signals are used. A pulsed, impulse-modulated direct current, whereby the frequency is between 1 and 30 Hz and the field strength is smaller than 25 gauss, preferably between 10 and 20 gauss, produces the electromagnetic signals. Through the cosmetic treatment of the skin with the portable applicator, in accordance with the invention, particularly the regeneration of collagen types II, IX, XI and X should be stimulated.

According to the invention, a device for use in pulsed signal therapy is provided. This device comprises an applicator, as described above in detail, and a controller for the feed of the control signal (control module) to the signal-generating units. A preferred embodiment sees the applicator and the control module as a compact unit. In this case, the applicator is closely connected to the control module; they may, for example, be screwed; together and/or the applicator and the control module may be an integral unit within a common frame. The module may be provided in a case or trolley, for ease of transportation.

According to another preferred embodiment, the control module is detachable from the applicator. This embodiment can also be provided within a case or trolley for ease of transportation.

During the operating period, the control module will feed the signal-generating units 20 with a control signal whereby the control signal is a pulsed impulse-modulated current with a rate of at most 30 pulses per second, preferably 1 to 12 pulses per second, and more preferably 5 to 12 pulses per second, and presents a rectangular pulse form with an abrupt increase and abrupt drop in voltage. As operation time passes, pulse width will diminish and pulse amount will increase. The term “pulse width”, as herein used, refers to pulse duration, whereas “pulse amount” refers to the number of pulses per time unit, whereby the data is given respectively per connection side.

Preferably, a cosmetic treatment will last around an hour per session, whereby the total time of the treatment will comprise 9 to 12 sessions. Optimally, the release of a control signal occurs throughout an 1-hour period, whereby in the first five minutes the largest pulse width and the lowest pulse amount will be released; from the sixth to the tenth minute, a smaller pulse width and a higher pulse amount are released; and from the eleventh to the sixtieth minute, the smallest pulse width and the highest pulse amount are released. Optimally, the pulse width of the first till fifth minute will be between 40-25 ms, and preferably 35-30 ms; from the sixth to the tenth minute 30-20 ms, and preferably 26-22 ms; and from the eleventh till the sixtieth minute 20-10 ms, and preferably 17-13 ms. The pulse amount will from the first till the fifth minute be 5 to 8, and in particular 6; from the sixth till the tenth minute 8 to 10, in particular 9; and from the eleventh till the sixtieth minute 10 to 12, in particular 11. Through the antidromic behaviour of the pulse width and pulse amount through time, the so-called duty cycle runs trough a maximum throughout the time; the maximum of the duty cycle appears between the sixth and the tenth minute in particular.

The duty cycle states the proportion of length of the activated status (pulse duration) to the cycle duration of a rectangular signal. The resulting value, multiplied by 100%, gives the duty cycle in percentage. In a preferred design, the duty cycle is from the first till the fifth minute 15-21%, in particular 17-20%; from the sixth to the tenth minute 20-25%, in particular 21-23%; and from the eleventh till the sixtieth minute 14-20%, in particular 15-17%.

The temperature of the skin is slightly raised due to the magnetic field produced, but not to a point at which the warmth of the skin becomes uncomfortable. The measured temperature values on the skin surface recorded after two treatments of one hour each with the facial mask of Design 2 of the present invention, for example, are as follows: middle upper lip circa 33° C., left and right cheeks circa 32° C., middle forehead circa 30° C.

The usage of the applicator described above for therapeutic purposes has also been taken into consideration. The term “therapeutic treatment of the skin”, as herein used, can comprise the treatment of wounds and scars for therapeutic purposes such as the acceleration or promotion of the healing of wounds, or the development of smaller or “smoother” scars. The therapeutic treatment of the skin is not limited to the examples described above, and may comprise all types of skin treatment for therapeutic purposes, whereby the stimulation of collagen regeneration may have positive effects on the appearance of the skin or on the healing process.

Besides the aforementioned cosmetic and therapeutic benefits of the invention in the treatment of skin, the portable applicator (or device) is equally suitable for a series of other medical uses. It may be used, for example, in the treatment of the following diseases: osteoporosis, carpal tunnel syndrome, tendonitis, fresh bone fractures, and stress-induced fractures, aseptic necrosis, fibromyalgia, Morton's syndrome, severe burns, epilepsy, migraines, and so on.

The embodiments described above are mere examples and the invention is by no means limited to them. The purpose of the present invention is defined as set forth by the following claims.

1. Portable applicator for pulsed signal therapy, with
a support body (10);
at least two signal-generating arrangements (30), which are positioned in or on the support body (10), whereby the at least two signal-generating arrangements (30) are connected in series, and each of the at least two signal-generating arrangements (30) includes at least two signal-generating units (20) which are connected in parallel.

2. Portable applicator according to claim 1, where each signal-generating arrangement (30) includes three signal-generating units (20) connected in parallel.

3. Portable applicator according to claim 1 or 2, wherein said signal-generating units (20) are cored coils.

4. Portable applicator according to any of the preceding claims, where the signal-generating units (20) produce a pulsed magnetic field with a field-strength of less than 25 G.

5. Portable applicator according to any of the preceding claims, where the signal-generating units (20) are imbedded in a protective body.

6. Portable applicator according to any of the preceding claims, where the support body (10) is surrounded by a coating.

7. Portable applicator according to claim 6, where the support body (10) is an insulating gel, in which the signal-generating arrangements (30) are imbedded.

8. Portable applicator according to any of the preceding claims, where the support body (10) is malleable.

9. Portable applicator according to any of the claims 1 to 7, where the support body (10) is built as a facial mask.

10. Portable applicator according to claim 8 or 9, where the support body (10) is built in several parts.

11. Portable applicator according to claim 10, where the several parts of the support body (10) are connected with each other in an articulated and/or elastic manner.

12. Use of an applicator according to any of the claims 1 to 11 for the cosmetic treatment of skin.

13. Use of an applicator according to any of the claims 1 to 11 for therapeutic treatment of skin.

14. Use according to claim 12 or claim 13, whereby the regeneration of collagen types II, IX, XI and X is stimulated.

15. Device for pulsed signal therapy, with an applicator according to any of the claims 1 to 11 and a device for the feed of the control signal to the signal-generating units (20).

16. Device according to claim 15, whereby the control signal is a pulsed direct current voltage with maximal rate of 30 pulses per second, preferably 1 to 12 pulses per second, and more preferably 5 to 12 pulses per second, and is a rectangular pulse form with an abrupt increase and an abrupt plunge in voltage.

17. Device according to claim 16, whereby the control module reduces the pulse broadness throughout the operating time and increases the pulse amount throughout the operating time.

18. Device according to any of the claims 15 to 17, where the control module is detachable from the applicator.
